(19) United States
(12) Patent Application Publication
(10) Pub. No.: US 2002/0127737 A1
(43) Pub. Date: Sep. 12, 2002

(54) TESTING DEVICE FOR DETERMINING ANALYTES IN A LIQUID DAIRY PRODUCT

(76) Inventors: JACQUES DEGELAEN, GENAPPES (BE); JEAN-MARIE FRERE, NANTRIN (BE); BENOIT GRANIER, ESNEUX (BE); BERNARD JORIS, SPA (BE)

Correspondence Address: WENDEROTH LIND & PONACK, 2033 K STREET NW SUITE 800, WASHINGTON, DC, 20006

(21) Appl. No.: 09/297,196

(22) PCT Filed: Oct. 6, 1998
(86) PCT No.: PCT/BE98/00147

(30) Foreign Application Priority Data
Oct. 7, 1997 (BE) 09700807
Jun. 25, 1998 (BE) 09800485

(51) Int. Cl. G01N 33/558

(52) U.S. Cl. 436/514

(57) ABSTRACT

The present invention relates to an assay device which allows the presence of analytes to be detected in a liquid dairy product by tangential capillary migration of the said dairy product, comprising a solid support (1) which has a first and a second end and on which the following membranes are fixed in succession starting from the first end:

a membrane (2) allowing the analysed liquid to be purified,

a membrane (3) on which one or more capture substances are immobilized, and

an absorbent membrane (4).

It also relates to a process allowing the detection and quantification of analytes in a liquid dairy product by virtue of this assay device and to an assay kit comprising this assay device.

The present invention relates to an assay device for determining analytes in a liquid dairy product such as milk. It also relates to a process allowing the detection and quantification of analytes in the milk, by virtue of this assay device, and to an assay kit comprising this assay device.

At present, sanitary requirements of numerous countries necessitate regular monitoring of the presence of various substances in dairy products, such as veterinary medicaments and hormones, which are commonly employed in the raising of cattle. For obvious medical reasons, these substances must be avoided in dairy products intended for human consumption.

In other cases, it is desirable to have tests which enable the presence of endogenous substances to be detected in milk in order to optimize the practices of cattle raising. In particular, rapid determination of the level of hormones in milk allows to identify readily favourable periods for reproduction.

In yet other cases, reliable and practical methods are sought for monitoring the origin of dairy products derived from the milk of various animal species. One then searches methods which would allow to identify the presence of proteins characteristic of the milk of certain species relative to others.

On the other hand, various tests are known in the literature for the detection of analytes in biological liquids. These tests generally use detection methods requiring a recognition agent (receptor or antibody), which recognizes specifically the analyte or an analogue of this analyte, and a labelling agent (radioelement, enzyme, fluorescent agent, etc.), hereinafter referred to as detection reagents. Depending on the elements chosen, such tests, may be called radioimmunoassay (RIA), radioreceptor assay (RRA), enzyme immunoassay (EIA), etc. In their general principle, these tests employ the minimum combination of the two above-mentioned elements (detection reagents) which will make it possible to obtain a result whose value is an indication of the quantity of analyte present.

It should be noted that, depending on the detection method selected, the labelling agent can be coupled alternatively to the recognition agent or to the analyte or to an analogue substance of the analyte in terms of its recognition by the recognition agent. There are also processes in which the recognition agent or the analyte or the analogue substance of the analyte contains, intrinsically, the labelling agent (for example, a radiolabelled analyte).

For dairy products, the analyte detection tests which are most widely described relate to the detection of antibiotics. It is indeed well known to use antibiotics for the treatment of certain infectious diseases of milk producing cattle.

However, for obvious medical reasons, milk intended for human consumption must, in principle, be exempt of any trace of antibiotic. On the other hand, penicillin concentrations of 0.005 I.U./ml or less may have negative impacts during the manufacture of dairy products such as cheese, yoghurt etc.

Various situations may be considered. Firstly, for example, in order to detect the presence of antibiotics at the farm before tanking into a truck, priority would be given to an extremely simple and quick text (less than 5 minutes). One may also consider using such a rapid test when, for example, the antibiotic use for the treatment is known and when, on the other hand, the test allows detection of the given antibiotic up to the legal standard. Secondly, when emphasis is not on rapidity, it is important to detect most, if not all, of the antibiotics at the legal standard requirement.

The reason for this is that the laws in certain countries impose quite specific quality standards. For example, the US authorities require that the concentrations in milk of the following six antibiotics do not exceed quite specific values: penicillin, 5 ppb; ampicillin, 10 ppb; amoxicillin, 10 ppb; cloxacillin, 10 ppb; cephapirin, 20 ppb, ceftiofur, 50 ppb. The European Union imposes quality standards as follows: penicillin, 4 ppb; amoxicillin, 4 ppb; ampicillin, 4 ppb; cloxacillin, 30 ppb; dicloxacillin, 30 ppb; oxacillin, 30 ppb; cephapirin, 10 ppb, ceftiofur, 100 ppb; cefquinone 20 ppb; nafcillin 30 ppb; cefazoline, 50 ppb.

It may thus be advantageous to have access to a test which would allow most of the antibiotics to be detected. Moreover, in the dairy industry, it may be considered that, in the absence of a test which has all the characteristics of speed, sensitivity and simplicity, a test which would allow the best combination of these three parameters, even if they are not totally covered, would be advantageous.

U.S. Pat. No. 4,239,852 describes a microbiological process for the detection in milk of antibiotics having a β-lactam ring. According to this process, the sample of milk is incubated firstly in the presence of cell parts of a microorganism which is highly sensitive to antibiotics, and especially Bacillus stearothermophilus, and secondly in the presence of an antibiotic which is labelled (“tagged”) with a radioactive element or with an enzyme. The incubation is conducted under conditions which allow antibiotics, if present in the sample, and the labelled antibiotic to bind to the cell parts.

Following incubation, the cell parts are separated from the mixture and then washed. Subsequently, the quantity of labelled antibiotic bound to the cell parts is determined and is compared with a standard. The quantity of labelled antibiotic bound to the cell parts is inversely proportional to the concentration of antibiotic present in the milk sample analysed.

This process requires fairly delicate handling, especially at the stage of separating the cell parts from the mixture. In addition, in its most sensitive version, this process uses an antibiotic labelled with a radioactive element (¹⁴C or ¹²⁵I). In this case, the determination of the quantity of antibiotic present or otherwise in the milk necessitates the use of a special instrument such as a scintillation counter, for example. In addition, handling radioactive products even in very small quantities is not completely free of risk for the person conducting the analysis.

European Patent Application 593 112 describes another method permitting the detection of antibiotics in milk. This method uses a protein isolated from an antibiotic-sensitive microorganism, such as Bacillus stearothermophilus. This protein is additionally labelled with an enzyme such as a peroxidase.

The test proceeds as follows: a sample of milk, is incubated in a tube in the presence of the labelled protein; after incubation, the milk is transferred to a second tube on whose walls a reference antibiotic has been immobilized; a second incubation is carried out, and then the contents of the tube are removed; the walls of this second tube are washed three times with a wash solution, which is itself removed, and then the residues present in the second tube are transferred to a piece of absorbent paper; a colouring substrate is then added to the second tube, which is incubated once again, and then a solution which stops the development of the colour is added; the coloration of the tube is compared with the coloration of an identical test carried out in parallel on a standard sample of antibiotic. The quantity of labelled protein immobilized on the support, and therefore the intensity of the coloration, is inversely proportional to the quantity of antibiotic present in the milk sample analysed.

According to Example 1 of this patent application, this test makes it possible to detect penicillin G down to concentrations of the order of 5 ppb and makes it possible to detect amoxicillin (5 ppb), ampicillin (10 ppb), cephapirin (5 ppb) and ceftiofur (5 ppb).

However, the test is very tiresome to carry out, especially by unskilled personnel. Indeed, this test comprises numerous steps, including steps of transferring liquid and residues from one vessel to another, and a number of rinsing steps. Given the number and the type of steps required in this test, obtaining a reliable result depends heavily on the experimental know-how of the operative.

In addition, interpreting the result requires two tests to be carried out in parallel, thereby multiplying and further complicating the operations.

Other types of enzymatic processes have also been disclosed, which make it possible to determine low concentrations of antibiotics in milk (J. M. Frere et al., Antimicrobial Agents and Chemotherapy, 18(4), 506-510 (1980), and patents EP 85 667 and EP 468 946), which are based on the use of a specific enzyme, namely soluble exocellular D-alanyl-D-alanine carboxypeptidase, which is produced by Actinomadura R39 (designated “enzyme R39” hereinafter). Enzyme R39 possesses a specific activity of hydrolysing the D-alanyl-D-alanine groups of various peptides and is also capable of hydrolysing specific thioesters.

In addition, enzyme R39 reacts with antibiotics having a β-lactam ring to form very rapidly an equimolar enzyme-antibiotic complex which is inactive and substantially irreversible.

In the most recent version of this test (EP 468 946) a predetermined volume of a sample of the liquid to be examined is incubated with a predetermined quantity of enzyme R39 under conditions which allow the β-lactam antibiotic which may be present in the sample to react with the enzyme to form an equimolar enzyme-antibiotic complex which is inactive and substantially irreversible.

Subsequently, a predetermined quantity of thioester-type substrate is incubated with the product obtained in the first stage under conditions which allow the substrate to be hydrolysed by the residual enzyme R39 which has not been complexed with the antibiotic in the course of the first incubation. The quantity of mercaptoalkanoic acid thus formed is then determined by colorimetric assay with the aid of a reagent capable of producing a coloration by reaction with the free SH group of the mercaptoalkanoic acid. The intensity of the coloration is compared with a standard established beforehand from samples containing known quantities of antibiotics. Quantitative determination can be carried out by measurement in a spectrophotometer; in the case of milk, it may be necessary to clarify the sample beforehand.

Clearly, this test involves fewer steps and is simpler to carry out than the test described in the EP Patent Application 593 112. However, it is limited to the detection of antibiotics having a β-lactam ring and to the threshold detection limits available with enzyme R39. Such as it is, this test cannot be used with other antibiotic receptors and cannot be used directly as a basis for detecting other analytes in milk.

Given that the present tests still have various disadvantages, the objective of the applicant was to search for new methods for the detection of analytes in liquid dairy products, the methods sought being required to enable different types of analytes to be determined reliably, preferably at the time of collection on the farm. The applicant has therefore researched a method which makes it possible to obtain, very rapidly, a reliable and sensitive result in a limited number of simple steps which do not require any special experimental know-how. In addition, the applicant has researched a method that supplies a result which can easily be detected visually and which can, moreover, be subjected to quantification by means of instrumental measurement.

The applicant has now discovered that these objectives can be obtained by virtue of the use of a novel assay device which makes it possible to determine with ease the presence of analytes in liquid dairy products, especially milk.

The present invention therefore provides an assay device which allows the presence of analytes to be detected in a liquid dairy product by tangential capillary migration of the said dairy product. The assay device according to the invention comprises a solid support (1) which has a first and a second end and on which the following membranes are fixed in succession starting from the first end:

a membrane (2) allowing the analysed liquid to be purified,

a membrane (3) on which one or more capture substances are immobilized, and

an absorbent membrane (4),

characterized in that the membrane (2) is capable of retaining the substances present in the dairy product which prevent the analytes, which may be present in the dairy product and the detection reagents used in accordance with the practised method, from migrating over the assay device during the tangential capillary migration of the sample after the first end of the assay device has been soaked in the analysed dairy product.

According to a particular embodiment of the invention, the assay device according to the present invention additionally possesses a membrane (5) on which at least one detection reagent has been deposited, this detection reagent being capable of solubilizing rapidly in the presence of the said dairy product. According to this particular embodiment, the membrane (5) must be placed before the membrane (3). It can be placed for example, alternatively in front of the membrane (2) at the first end of the device, or between the membrane (2) and the membrane (3), or else above or below the membrane (2).

The different membranes present in the assay device according to the present invention are superimposed on one another at their ends so as to ensure the continuous migration of the dairy product from one zone to the other. Preferably, the membrane (3) is located such that its proximal end is located below the membrane (2) and its distal end below the membrane (4). The membranes can optionally be held in contact with one another by virtue of an adhesive plastic film (6). In this case, the adhesive plastic film is selected so as not to affect the migration of the liquid over the assay device.

The option of covering the assay device with an adhesive plastic film has two advantages: it ensures perfect contact at the point of superimposition of the membranes, and constitutes a protective film. The adhesive plastic film (6) can either cover the membranes (2), (3), (4) and (5) completely or partially cover the individual membranes. Preferably, the adhesive plastic film (6) does not cover the first few millimeters of the first end, in order to allow more rapid migration of the liquid over the membrane (2) of the assay device.

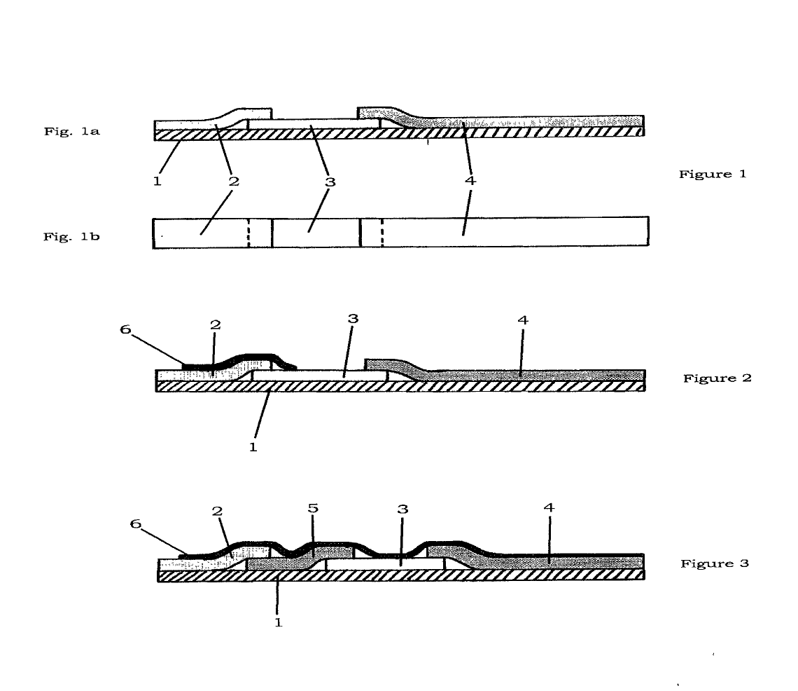

FIGS. 1 to 3 illustrate examples of assay devices according to the present invention. FIGS. 1a, 2 and 3 show front views, and FIG. 1b shows a view in longitudinal section.

The solid support (1) present in the assay device according to the present invention is made of glass or plastic, preferably plastic. In the case of a support made of plastic its thickness is generally between 0.05 and 1 mm, preferably between 0.1 and 0.6 mm. The membranes are Fixed on the solid support (1) by means of an adhesive.

The membrane (2) can be of various types. On the one hand, it must allow to retain the substances present in the dairy product which prevent the analytes which may be present in the dairy product and the detection reagents used in accordance with the practised method from migrating over the assay device. On the other hand, it must allow rapid migration of analytes and detection reagents on the assay device, while preserving the activity of these analytes and detection reagents during said migration. Non-limiting examples of membranes which make it possible to obtain this result are: Cytosep 1663 and Leukosorb LK4 (available from Pall Gelman Sciences), GF/D, GF/DVA, 17 CHR (available from Whatmann) and fibre glass type GF141 (available from Alstrom).

The Leukosorb membrane preferably used is a membrane manufactured from non-woven polyester fibres and is intended for retaining leukocytes from clinical samples obtained from blood, urine, saliva and the cerebrospinal fluid. Leukocyte retention is realised by filtration of the fluid by transversal passage through the membrane.

The applicant has discovered, unexpectedly, that these types of membranes also make it possible to provide a very important function for the detection of analytes in a dairy product by virtue of the assay devices according to the present invention, namely the retention of substances, present in the dairy products, which prevent the proper functioning of a test of detection by tangential capillary migration of the dairy product over the said assay devices, while allowing at the same time rapid migration of the analytes and detection reagents.

In order to carry out this function to best effect, the membrane (2) must be sufficiently long to allow the removal of all of the substances present in the dairy product which prevent the migration of the analytes and detection reagents over the assay device.

The membrane (3) must make it possible to immobilize one or more capture substances and must allow rapid migration of the dairy product sample after migration over membrane (2). Preferably, the membrane (3) is consolidated on a nonporous support of the polyester type. Non-limiting examples of membranes suitable according to the present invention are: high-tangential speed nitrocellulose membranes, such as Hi-Flow membranes (available from Millipore), preferably Hi-Flow membranes of types SX or ST. The membranes provide an optimal result in combination with membranes (2) identified above.

One or several capture substances are immobilised on the membrane (3). The type of capture substance immobilised depends, of course, on the practised method for the detection of the analytes; the capture substances are capable of selectively immobilizing at least one of the constituents present in the liquid which migrates over the assay device, such as one of the detection reagents, or the product resulting from the formation of a complex between the analyte, or an analogue substance of this analyte, and at least one detection reagent or, alternatively, the product resulting from the formation of a complex between two or more detection reagents. The capture substance can also be one of the detection reagents. The capture substances are located in concentrated form on a portion or several well-defined portions of the membrane (3), such as in discs or thin strips, or any other design appropriate for the application. Whatever the design chosen, it must allow clear reading of the result.

As far as its dimensions are concerned, the membrane (3) must be long enough to contain all of the capture substances used in the order and in the quantities required in accordance with the practised method of detection.

The type of membrane used for the membrane (4) is of minor importance provided that it is capable of absorbing and storing the liquid after its passage over the preceding membranes. The membrane (4) is sufficiently large to allow it to absorb the liquid after its passage over the membrane (3) but also, from a practical viewpoint, to allow the assay device to be handled more easily.

Optionally, the assay device according to the present invention may include a membrane (5) on which at least one detection reagent has been deposited. The membrane (5) must allow the migration of the dairy product while at the same time allowing the solubilization and release of the detection reagent (or reagents) upon the passage of flow of dairy product. Non-limiting examples of membranes which may be suitable for this purpose are: glass fibre resin-based membranes, such as T5NM membranes (available from Millipore), F075-14 or F075-17 or GF/C membranes (available from Whatman), the membrane Cytosep 1663 (available from Pall Gelman Sciences), polyester fibre resin-based membranes, such as Accuwick membranes (available from Pall Gelman Sciences), 3 mm cellulosic paper (available from Whatman) or else membranes of the Release Matrix PT-R2 type (available from Advanced Micro Devices). It is preferred to use a polyester fibre resin-based membrane such as the Accuwick membranes. The membrane (5) is long enough to support the desired quantity of detection reagent.

The assay devices according to the present invention are manufactured by the methods known to the skilled in the art. Cards can be prepared, for example, using commercially available laminators. The capture substances used are deposited on the membrane (3) in the form of solutions, before or after the assembly of the cards. These solutions can be deposited very precisely using commercially available apparatus such as the BioJet Quanti3000 X-Y platform dispenser from BioDot, Inc. These deposited solutions are immediately evaporated, for example, by placing the card under a stream of hot air. For large-scale production it is also possible to prepare rolls. Subsequently, the cards and rolls bearing the desired capture substances are cut into strips, each of these strips constituting an assay device according to the invention.

When the assay device includes a membrane (5), the detection reagent(s) can be deposited thereon prior to the assemble of the cards or rolls by simply immersing the membrane (5) in a solution containing the detection reagent(s). Alternatively, the reagent(s) can be deposited after the assembly of the cards or rolls, by a technique similar to that used for depositing the capture substances on the membrane (3).

In one variant of the invention, the device is located in a plastic box which has two apertures: the first, in the form of a basin, is located just above the membrane (2) and admits the liquid to be analysed; the second is a window aperture allowing the result on the membrane (3) to be observed. In this case, the assay device does not include an adhesive plastic film (6).

The assay device according to the present invention allows the detection of the presence of analytes in a liquid dairy product, especially milk.

Consequently, the present invention likewise provides a process for detecting analytes in a liquid dairy product, using an assay device according to the present invention, and detection reagents, and comprising the following steps:

a) the bringing into contact of a defined volume of dairy product with the assay device according to the present invention, this contact taking place at the first end of the assay device,

b) the tangential migration, by capillarity, of the dairy product over the assay device such that the analytes and the detection reagents which may be present in the dairy product pass gradually over the membrane (2), then the membrane (3), and such that the constituents of the dairy product which are not stopped by the membranes (2) and (3) end up in the membrane (4), and

c) the determination of a fixation on the membrane (3).

The process according to the present invention allows the detection of analytes in a liquid dairy product such as, for example, milk, whey, churned milk, etc. The present invention is directed more particularly to the detection of analytes such as veterinary medicaments, hormones or proteins which may be present in milk.

The detection reagents used according to the process of the invention may vary in number and in nature as a function of the mode of implementation of the invention, which is itself based on the method of detection practised. The process according to the invention uses at least two detection reagents. The first detection reagent is a recognition agent capable of recognizing specifically the analyte and will be referred to below as “identifier”. The second detection reagent is a labelling agent and will be referred to below as “marker”. It should be noted that, depending on the selected mode of implementation, certain detection reagents may be present on the membrane (3) or on the membrane (5). Depending on the analyte to be detected and on the detection reagents used, it may prove necessary to add one or more detection reagents before the step of bringing the dairy product into contact with the assay device according to the invention and to maintain this mixture under incubation conditions which allow the formation of a complex between the detection reagents and the analyte or an analogue substance of the analyte. Depending on the selected method, the identifier and the marker can be coupled to one another or can be a single substance. On the other hand, there may be a plurality of identifiers and/or markers.

The identifier allows the detection of the presence of the type of analyte sought by virtue of its capacity to recognize specifically this analyte or an analogue substance of this analyte. It may be a receptor which is able to form selectively a stable and essentially irreversible complex with the analyte or an analogue substance of the analyte or a monoclonal or polyclonal antibody specific for the analyte or for an analogue substance of the analyte. For the detection of antibiotics, the identifier can be selected from specific polyclonal or monoclonal antibodies or from the receptors obtained from microorganisms sensitive to the antibiotics, such as the receptors obtained from Bacillus species (Bacillus stearothermophilus, Bacillus subtilis, Bacillus licheniformis, etc.), Streptococcus species (Streptococcus thermophilus, etc.), or Actinomycetes species (Actinomadura R39, etc.).

According to a preferred embodiment of the invention an identifier is used which comprises a receptor sensitive to antibiotics having a β-lactam ring, which receptor is obtained from Bacillus licheniformis, such as the receptor BlaR or the receptor BlaR-CTD. The isolation and the peptide sequence of the protein BlaR are described in Y. Zhu et al., J. Bacteriol., 1137-1141 (1990); the receptor BlaR-CTD is the carboxy-terminal region of BlaR, whose isolation and peptide sequence are described in B. Joris et al., FEMS Microbiology Letters, 107-114 (1990).

The use of the receptors BlaR or BlaR-CTD according to the present intention for the detection of antibiotics having a β-lactam ring has major advantages over the recognition agents used to date. In fact, the receptors BlaR and BlaR-CTD are capable very rapidly of complexing a large number of antibiotics and of doing so at an incubation temperature which is less than that required for the known recognition agents such as, for example, the receptors obtained from Bacillus stearothermophilus.

The second type of detection reagent used is a marker, which allows visualization and direct or indirect quantification of the presence of the analytes in the dairy product. The markers which can be used according to the invention can be particulate, fluorescent, radioactive, luminescent or enzymatic. It is preferred to select a particulate marker, which gives a readily detectable, visual signal even when present in a small quantity. As non-limiting examples mention may be made of colloidal metallic particles (platinum, gold, silver, etc.), colloidal particles of selenium, carbon, sulphur, tellurium, or else coloured, synthetic, colloidal particles of latex. Colloidal gold particles having a diameter of between 1 and 60 nm are preferred; they give a readily detectable, intense pink-red coloration.

The marker makes it possible to determine the presence of the analyte in the sample of dairy product by virtue of its coupling with one or more detection reagents, with the analyte or with an analogue substance of the analyte.

Coupling between the marker and the detection reagent can be carried out in accordance with methods known to the skilled worker. When a particulate marker is used, labelling can take place either by direct adsorption on the particles or indirectly, via the intermediacy of a chemical anchoring arm such as, for example, a biotin/anti-biotin complex. This coupling may take place either before the stage of bringing the dairy product into contact with the assay device according to the invention or during the migration of the dairy product over the assay device according to the invention.

According to one particular embodiment of the invention a third type of detection reagent is used, referred to hereinafter as “reference”. This is a substance added in a known quantity to the sample analysed, which fixes itself to a specific capture substance immobilized on the membrane (3). The reference gives a band whose intensity serves as a reference for quantifying the analyte.

As far as the bringing of the dairy product into contact with the assay device according to the invention is concerned (step a) of the process), it is carried out by placing the assay device according to the present invention in a vessel at whose bottom there is the sample to be analysed. The assay device is placed essentially vertically in the vessel, such that the first end of the device is in contact with the mixture.

In the variant of the invention using an assay device located in a plastic box, the box is arranged horizontally and contact takes place by depositing an aliquot of the sample to be analysed in the basin-shaped opening located above the membrane (2).

For the migration step b), the liquid is allowed to migrate by capillarity over the assay device according to the invention. The liquid which migrates by capillarity over the assay device according to the invention first meets the membrane (2), which makes it possible to retain those substances present in the dairy product which prevent the migration of analytes which may be present in the dairy product and detection reagents over the assay device. The analytes and detection reagents subsequently migrate over the membrane (3) on which one or more capture substances have been immobilized. The capture substances selectively immobilize at least one of the constituents present in the analysed liquid. According to a particular embodiment of the invention use is made of a capture substance, located at the end of the migration path of the liquid over the membrane (3), which is capable of fixing all of the markers which have not been stopped by the preceding capture substances. This capture substance makes it possible to supplement fully the quantitative information supplied by the preceding capture substances.

The determination of a fixation on the membrane (3) (step c) of the process) is carried out simply by determining the presence of markers in this zone. This determination is possible, in a simple manner, visually. However, if precise measurement of the intensity of the observed signals is desired it is possible to employ an instrument capable of measuring the intensity of the observed signal. When a reference is used, it is fixed by a specific capture substance which supplies an internal reference for the measurement of the intensity of the signals observed.

The interpretation of the result obtained depends on the method of detection practised, namely on the detection reagents and capture substances that are employed.

In relation to the processes for detecting analytes in milk that have been described previously in the literature, the process according to the present invention has the following advantages. Firstly, this process is very rapid and extremely simple to implement: it comprises essentially two easy operating steps requiring no special experimental know-how. Subsequently, the qualitative and quantitative appraisal of the result is immediate and does not require special additional operations, such as those required when detection is carried out by way of colorants and/or enzymatic markers. In addition, this process can be applied directly to the detection of different types of analytes. Finally, in the embodiment using a reference, the result can be directly quantified and interpreted without the need to carry out one or more reference tests.

The present invention also provides an assay kit for the detection of analytes in a dairy product, comprising an assay device according to the present invention. If appropriate, the assay kit according to the present invention may also include detection reagents for addition to the sample before the dairy product is brought into contact with the assay device.

The examples which follow illustrate various aspects and embodiments of the present invention without, however, limiting its scope.

### EXAMPLE 1

### Manufacture of Assay Devices. General Procedures

1.1. Assembling membrane cards.

Cards having a size of 300×76.2 mm are first of all assembled using a laminator of the Chlamshell Laminator type (available from BioDot, Inc.) in accordance with the following method:

A plastic support rectangle of type ArCare 8565 (available from Adhesive Research) is cut out, measuring 300×76.2 mm (solid support (1)). Subsequently, a rectangle of Leukosorb LK4 membrane (available from Pall Gelman Sciences), measuring 300×20 mm (membrane (2)), a rectangle of Hi-Flow SX membrane (available from Millipore), measuring 300×25 mm (membrane (3)), a rectangle of 3 mm cellulose membrane (available from Whatman), measuring 300×40 mm (membrane (4)) and a rectangle of Accuwick membrane (available from Pall Gelman Sciences), measuring 300×0.8 mm (membrane (5)), are cut out.

In succession, the membranes (2) and (4), then (5), then (3) are placed in a specific location of the lower mould of the laminator. The solid support (1), covered with adhesive, is for its part held in the cover of the apparatus, with the adhesive face exposed to the air. The membranes placed in the lower mould are brought into contact with the adhesive support by closing the laminator; the membranes are held exactly in place by means of air suction from a vacuum pump. When the vacuum is broken, a card is recovered which consists of the solid support (1) with, fixed thereon, the membranes (2), (3), (4) and (5).

1.2. Depositing capture substances on the membrane (3).

The deposition of the capture substances on the membrane (3) is carried out before or after the assembly according to Example 1.1.

An aqueous solution containing the capture substance is prepared. It is deposited on the membrane (3) of the membrane card prepared in Example 1.1. by means of a BioJet X-Y Platform Quanti3000 Dispenser from BioDot, Inc.

The deposited solutions are immediately evaporated by placing the whole of the card under a stream of hot pulsed air at 60° C. for one minute.

1.3. Depositing the labelling substance on the membrane (5)

a) Before the assembly according to Example 1.1.

An aqueous solution containing the labelling substance is prepared. The membrane (5) is immersed in this solution. It is subsequently drained and then dried overnight at ambient temperature under a vacuum of 0.5 bar.

b) After the assembly according to Example 1.1.

The procedure described in Example 1.2. for the deposition of the capture substances is followed.

1.4. Depositing the adhesive plastic film (6).

A rectangle of adhesive film of the type ArCare 7759 (available from Adhesive Research) is cut out, measuring 300×20 mm for partial covering and 300×71.2 mm for covering all the membranes.

The card obtained according to Example 1.1. is placed in the lower mould of a laminator and the adhesive film is placed in the cover of the laminator, with the adhesive face exposed to the air. The adhesive plastic film is brought into contact with the membrane card when the apparatus is closed.

1.5. Cutting into strips.

The cards obtained after assembly are cut into strips with the aid of a guillotine-type apparatus or with the aid of a rotary apparatus (available from BioDot, Kinematic or Akzo). The end strips are removed, with the other strips being ready for use.

To preserve them, the assay devices are placed in an opaque, hermetically sealed container in the presence of a dessicant (Silgelac, France).

1.6. Presentation in a plastic case.

The assay device is placed in a plastic box which has two openings: the first, in the form of a basin, is situated just above the membrane (2) and makes it possible to receive the liquid to be analysed; the second is a window aperture which allows the result on the membrane (3) to be visualized.

### EXAMPLE 2

### Detection of Antibiotics Having a β-lactam Ring in Milk, using Enzyme R39

2.1. Identifier.

The identifier used in this example is soluble exocellular D-alanyl-D-alanine carboxypeptidase produced by Actinomadura R39, obtained by the procedure described in J.-M. Frere et al., Antimicrobial Agents and Chemotherapy, 18(4), 506-510 (1980).

2.2. Coupling the identifier with the marker.

2.2.1. Biotinylating the identifier.

250 μl of an aqueous solution of enzyme R39 having a concentration of 1 mg/ml are dialysed for 24 hours against 500 ml of HNM buffer (Hepes 10 mM, pH 8, NaCl 10 mM, MgCl₂ 5 mM). To this dialysed solution of enzyme R39 are then added 2 ml of bicarbonate buffer (0.1M sodium bicarbonate, pH 9) and 250 μl of a solution of N-hydroxysuccinimide 6-(biotinamido)caproic ester having a concentration of 5 mg/ml in anhydrous DMF. This solution is stirred gently in the LABINCO stirrer for tubes on a rotary axis (available from VEL, Belgium) at a rate of 2 revolutions/minute for 3 hours at room temperature and away from light. The solution thus obtained is dialysed against the HNM buffer (Hepes 100 mM; pH 8, NaCl 100 mM, MgCl₂ 50 mM) for 24 hours. In this way a solution of biotinylated enzyme R39 is obtained which is diluted in HNM-BSA buffer (Hepes 500 mM; pH 8, NaCl 500 mM, MgCl₂ 250 mM, BSA 10 mg/ml) to a concentration of 100 μg of enzyme R39 per ml of buffer. This solution is stored at −20° C.

2.2.2. Marker.

As the marker use is made of particles of gold having a diameter of 40 nm on which a goat anti-biotin antibody has been deposited in the form of suspensions in a 2 mM aqueous sodium tetraborate solution, with a pH of 7.2, stabilized by 0.1% of sodium azide (available from British Biocell (Ref. GAB40)). The optical density of these suspensions at 520 nm is approximately 10 and the protein concentration is approximately 24 μg/ml.

2.2.3. Coupling the biotinylated identifier with the marker.

Solution A for rapid test

The biotinylated enzyme R39 solution prepared in Example 2.2.1 is diluted 25 times with the HNM-BSA buffer (Hepes 500 mM, pH 8, NaCl 500 mM, MgCl₂ 250 mM, BSA 10 mg/ml). At room temperature, 17.5 parts by volume of this dilute biotinylated enzyme R39 solution, 9.27 parts by volume of the gold particle suspension used to label the enzyme R39 and 6 parts by volume of reference gold particle suspension are mixed (see Example 2.3).

Solution B for sensitive test

The biotinylated enzyme R39 solution prepared in Example 2.2.1 is diluted 50 times with the HNM-BSA buffer (Hepes 500 mM, pH 8, NaCl 500 mM, MgCl₂ 250 mM, BSA 10 mg/ml). At room temperature, 17.5 parts by volume of this dilute biotinylated enzyme R39 solution, 9.27 parts by volume of the gold particle suspension used to label the enzyme R39 and 6 parts by volume of reference gold particle suspension are mixed (see Example 2.3).

2.3. Reference

As the reference use is made of 40 nm particles of gold on which a goat anti-rabbit immunoglobulin antibody has been deposited. These particles are available from British Biocell (Ref. GAR40) in the form of suspensions in 2 mM aqueous sodium tetraborate solution, with a pH of 7.2, stabilized by 0.1% sodium azide. The optical density of these suspensions at 520 nm is approximately 3 and the protein concentration is approximately 6 μg/ml.

2.4. Capture substances.

2.4.1. First capture substance.

8 ml of a solution containing 213 mg of human gamma globulin (G4386, Sigma) and 8.6 mg of 2-iminothiolane hydrochloride (Aldrich, 33056-6) in sodium carbonate buffer (100 mM, pH 9) are incubated at 25° C. for one hour.

In addition, 20 ml of a solution containing 119.8 mg of cephalosporin C and 54 mg of sulphosuccinimidyl 4-(N-maleimidomethyl)cyclohexane-1-carboxylate (sSMCC, 22322 Pierce) in sodium carbonate buffer (100 mM, pH 9) are incubated at 25° C. for one hour.

The two solutions prepared above are then mixed. The pH of the resulting solution is adjusted to 7.1 by adding 3 ml of NaH₂PO₄ 500 mM, and the solution is incubated at 25° C. for two hours. The mixture obtained after incubation is dialysed three times against 1 liter of sodium phosphate buffer (10 mM, pH 7.5). The resulting solution is filtered through a 0.22 mm filter, then divided into aliquots and frozen at −20° C. until use.

At the time of use, the aliquots are thawed and a food colorant is added to them before they are deposited on the membrane, so as to indicate at any moment the exact position of the deposit and the quality of the trace.

The first capture substance makes it possible to fix the identifiers coupled with the free markers present in excess relative to the quantity of antibiotic present in the sample.

2.4.2. Second capture substance.

For the second capture substance use is made of a rabbit immunoglobulin solution (Sigma I 5006) having an immunoglobulin concentration of 0.5 mg/ml in a 10 mM sodium phosphate, pH 7.5, human gamma globulin 5 mg/ml buffer. This second capture substance stops the reference as the liquid migrates over the assay device.

2.5. Assay device.

Assay devices containing membranes (2), (3) and (4) are used, assembled in accordance with the procedure described in Example 1.1. The membrane (3) of these devices carries on the proximal side the capture substance described in Example 2.4.1. and on the distal side the capture substance described in Example 2.4.2. The capture substances were deposited in accordance with the procedure described in Example 1.2.

2.5.1. Test 1—Rapid test.

Seven samples of milk are prepared, containing 0; 2; 4; 5; 6; 8 and 10 ppb, respectively, of penicillin G. Each of these solutions is then analysed as follows.

An aliquot is taken of 200 μl of milk sample and 32.8 ml of solution A prepared in Example 2.2.3, and is placed in an Eppendorf tube. This mixture is incubated at 47° C. for 3 minutes. An assay device is then placed vertically in the Eppendorf tube such that the first end of the assay device is in contact with the mixture. The mixture is allowed to migrate over the assay device while the assembly is incubated at 47° C. for 2 minutes.

Table 1 below shows the results obtained for the 7 samples tested. An intensity value ranging from 0 to 10 is awarded to the bands detected, the value 10 being given to the most intense band and the value 0 being given to the least intense band. On this scale, a value 6 is awarded to the reference band. The intensity of the signal observed in the first detection band is inversely proportional to the quantity of penicillin G present in the sample.

TABLE 1

| | |
|---|---|
| Penicillin G | Intensity |

| (ppb) | 1st band | 2nd band |
|---|---|---|
| 0 | 10 | 6 |
| 2 | 8 | 6 |
| 4 | 5 | 6 |
| 5 | 3 | 6 |
| 6 | 2 | 6 |
| 8 | 1 | 6 |
| 10 | 0 | 6 |

In this example, the test is considered to be positive when the first band has an intensity which is lower than that of the reference band. The results shown in Table 1 indicate that this test makes it possible to detect in 5 minutes down to 4 ppb of penicillin G in a milk sample.

2.5.2. Test 2—Sensitive test.

Six samples of milk are prepared, containing 0; 2; 2.5; 3; 4 and 5 ppb, respectively, of penicillin G. Each of these solutions is then analysed as follows.

An aliquot is taken of 200 μl of milk sample and 32.8 μl of solution B prepared in Example 2.2.3, and is placed in an Eppendorf tube. This mixture is incubated at 47° C. for 5 minutes. An assay device is then placed vertically in the Eppendorf tube such that the first end of the assay device is in contact with the mixture. The mixture is allowed to migrate over the assay device while the assembly is incubated at 47° C. for 2 minutes.

Table 2 below shows the results obtained for the 6 samples tested. An intensity value ranging from 0 to 10 is awarded to the bands detected, the value 10 being given to the most intense band and the value 0 being given to the least intense band. On this scale, a value 6 is awarded to the reference band. The intensity of the signal observed in the first detection band is inversely proportional to the quantity of penicillin G present in the sample.

TABLE 2

| | |
|---|---|
| Penicillin G | Intensity |

| (ppb) | 1st band | 2nd band |
|---|---|---|
| 0 | 10 | 6 |
| 2 | 7 | 6 |
| 2.5 | 5 | 6 |
| 3 | 4 | 6 |
| 4 | 1 | 6 |
| 5 | 0 | 6 |

In this example, the test is considered to be positive when the first band has an intensity which is lower than that of the reference band. The results shown in Table 2 indicate that this test makes it possible to detect in 7 minutes down to 2.5 ppb of penicillin a milk sample.

### EXAMPLE 3

### Determination of Antibiotics Having a β-lactam Ring in Milk, using BlaR

This example illustrates the detection in milk of antibiotics having a β-lactam ring which are monitored by the health authorities. The test described in this example uses the receptor BlaR-CTD coupled with gold beads, which serve as labelling agents, and uses a support which is in the form of an assay device comprising a solid support on which membranes are fixed.

3.1. Coupling of BlaR-CTD (identifier) with the gold beads (marker).

3.1.1. Biotinylation of BlaR-CTD.

3.79 ml of a solution of recognition agent BlaR-CTD having a concentration of 6.6 mg/ml are taken up in a sodium phosphate buffer, 20 mM pH 7. To this solution of BlaR-CTD are then added 41.71 ml of bicarbonate buffer (0.1M sodium bicarbonate, pH 9) and 2 ml of a solution of N-hydroxysuccinimide 6-(biotinamido)caproic ester containing 2.23 mg/ml likewise of bicarbonate buffer. This solution is stirred gently on a LABINCO stirrer for tubes on a rotary axis (available from VEL, Belgium) at a rate of 2 revolutions/minute for 2 hours at ambient temperature and away from light. 2.5 ml of a solution of Tris buffer, 1M pH 8 are incubated with the reaction mixture under the same conditions for 30 minutes. The solution thus obtained is dialysed against HNM buffer (Hepes 100 mM, pH 8, NaCl 100 mM, MgCl₂ 50 mM) for 24 hours. In this way a biotinylated BlaR-CTD solution is obtained which is diluted in HNM-BSA buffer (Hepes 500 mM, pH 8, NaCl 500 mM, MgCl₂ 250 mM, BSA 10 mg/ml) to a concentration of 250 μg of biotinylated BlaR-CTD per ml of buffer. This solution is stored at −20° C.

3.1.2. Labelling agent.

As the labelling agent use is made of particles of gold having a diameter of 40 nm on which a goat anti-biotin antibody has been deposited in the form of suspensions in a 2 mM aqueous sodium tetraborate solution, with a pH of 7.2, stabilized by 0.1% of sodium azide (available from British Biocell (Ref. GAB40)). The optical density of these suspensions at 520 nm is approximately 10 and the protein concentration is approximately 24 μg/ml.

3.1.3. Coupling the biotinylated BlaR-CTD to gold beads.

The biotinylated BlaR-CTD solution prepared in Example 3.1.1 is diluted 114.7 times with the HNM-BSA buffer (Hepes 500 mM, pH 8, NaCl 500 mM, MgCl₂ 250 mM, BSA 10 mg/ml). At room temperature, 22.5 parts by volume of this dilute biotinylated BlaR-CTD solution, 7.5 parts by volume of HNM-BSA buffer, 9.27 parts by volume of the gold particle suspension used to label the biotinylated BlaR-CTD and 6 parts by volume of reference gold particle suspension are mixed (see Example 3.1.4 below).

3.1.4. Independent reference.

In this test, use is also made of a reference substance which supplies a band whose intensity enables rapid quantification of the antibiotic present in the sample.

For this purpose use is made of 40 nm particles of gold on which a goat anti-rabbit immunoglobulin antibody has been deposited. These particles are available from British Biocell (Ref. GAR40) in the form of suspensions in 2 mM aqueous sodium tetraborate solution, with a pH of 7.2, stabilized by 0.1% sodium azide. The optical density of these suspensions at 520 nm is approximately 3 and the protein concentration is approximately 6 μg/ml.

3.2. Capture substances.

3.2.1. First capture substance—reference antibiotic.

8 ml of a solution containing 213 mg of human gamma globulin (G4386, Sigma) and 8.6 mg of 2-iminothiolane hydrochloride (Aldrich, 33056-6) in sodium carbonate buffer (100 mM, pH 9) are incubated at 25° C. for one hour.

In addition, 20 ml of a solution containing 119.8 mg of cephalosporin C and 54 mg of sulphosuccinimidyl 4-(N-maleimidomethyl)cyclohexane-1-carboxylate (sSMCC, 22322 Pierce) in sodium carbonate buffer (100 mM, pH 9) are incubated at 25° C. for one hour.

The two solutions prepared above are then mixed. The pH of the resulting solution is adjusted to 7.1 by adding 3 ml of NaH₂PO₄ 500 mM, and the solution is incubated at 25° C. for two hours. The mixture obtained after incubation is dialysed three times against 1 liter of sodium phosphate buffer (10 mM, pH 7.5). The resulting solution is filtered through a 0.22 μm filter, then divided into aliquots and frozen at −20° C. until use.

At the time of use, the aliquots are thawed and a food colorant is added to them before they are deposited on the membrane, so as to indicate at any moment the exact position of the deposit and the quality of the trace.

The first capture substance makes it possible to fix the BlaR-CTD coupled with the gold beads present in excess relative to the quantity of antibiotic present in the sample.

3.2.2. Second capture substance—substance capable of fixing the independent reference.

For the second capture substance use is made of a rabbit immunoglobulin solution (Sigma I 5006) having an immunoglobulin concentration of 0.5 mg/ml in a 10 mM sodium phosphate, pH 7.5, human gamma globulin 5 mg/ml buffer. This second capture substance stops the independent reference as the liquid migrates over the assay device.

3.3. Assay device.

Assay devices containing membranes (2), (3) and (4) are used, assembled in accordance with the procedure described in Example 1.1. The membrane (3) of these devices carries on the proximal side the capture substance described in Example 3.2.1. and on the distal side the capture substance described in Example 3.2.2. The capture substances were deposited in accordance with the procedure described in Example 1.2.

3.4. Determination of antibiotics in milk.

3.4.1. 3-minute test—rapid test.

7 samples of fresh milk are prepared, containing 0; 1; 2; 3; 4; 5 and 6 ppb, respectively, of penicillin G. Each of these solutions is then analysed as follows.

An aliquot is taken of 200 μl of milk sample and 45.27 μl of solution prepared in Example 3.1.3, and is placed in a glass flask. This mixture is incubated at 47° C. for 1 minute. An assay device is taken and is placed vertically in the glass flask such that the first end of the assay device is in contact with the mixture and such that the second end rests on the wall of the glass flask. The mixture is allowed to migrate over the assay device while the assembly is incubated at 47° C. for 2 minutes.

Table 1 below shows the results obtained for the 7 samples tested. An intensity value ranging from 0 to 10 is awarded to the bands detected, the value 10 being given to the most intense band and the value 0 being given to the least intense band. On this scale, a value of 6 is awarded to the reference band. The intensity of the signal observed in the first detection band is inversely proportional to the quantity of penicillin G present in the sample.

TABLE 1

| | |
|---|---|
| Penicillin G | Intensity |

| (ppb) | 1st band | 2nd band |
|---|---|---|
| 0 | 10 | 6 |
| 1 | 9 | 6 |
| 2 | 9 | 6 |
| 3 | 4 | 6 |
| 4 | 0 | 6 |
| 5 | 0 | 6 |
| 6 | 0 | 6 |

In this example, the test is considered to be positive when the first band has an intensity which is lower than that of the second band. The results shown in Table 1 indicate that this test makes it possible to detect in 3 minutes less than 4 ppb of penicillin G in a milk sample.

Assays were also carried out with other β-lactam ring antibiotics under the same conditions. This test, carried out in 3 minutes, makes it possible to detect amoxycillin down to 5 ppb, ampicillin down to 5 ppb, cloxacillin at less than 10 ppb, dicloxacillin at less than 20 ppb, oxacillin at less than 20 ppb and cephapirin down to 20 ppb in a milk sample.

1.3.2. Five minute test

Six samples of fresh milk are prepared, containing 0; 2; 4; 6; 8 and 10 ppb, respectively, of cloxacillin. Each of these solutions is then analysed as follows.

An aliquot is taken of 200 μl of milk sample and 45.27 μl of solution prepared in Example 3.1.3, and is placed in a glass flask. This mixture is incubated at 47° C. for 3 minutes. An assay device is taken and is placed vertically in the glass flask such that the first end of the assay device is in contact with the mixture in such a way that the second end rests on the wall of the glass flask. The mixture is allowed to migrate over the assay device while the assembly is incubated at 47° C. for 2 minutes.

Table 2 below shows the results obtained for the 6 samples tested. An intensity value ranging from 0 to 10 is awarded to the bands detected, the value 10 being given to the most intense band and the value 0 being given to the least intense band. On this scale, a value of 6 is awarded to the reference band. The intensity of the signal observed in the first band is inversely proportional to the quantity of cloxacillin present in the sample.

TABLE 2

| | |
|---|---|
| Cloxacillin | Intensity |

| (ppb) | 1st band | 2nd band |
|---|---|---|
| 0 | 10 | 6 |
| 2 | 6 | 6 |
| 4 | 5 | 6 |
| 6 | 3 | 6 |
| 8 | 3 | 6 |
| 10 | 3 | 6 |

In this example, the test is considered to be positive when the first band has an intensity which is lower than that of the second band. The results shown in Table 2 indicate that this test makes it possible to detect in 5 minutes down to 4 ppb of cloxacillin in a milk sample.

Assays were also carried out with other β-lactam ring antibiotics under the same conditions. This test, carried out in 5 minutes, makes it possible to detect penicillin G down to 3 ppb, amoxicillin down to 4 ppb, ampicillin down to 4 ppb, dicloxacillin down to 8 ppb, oxacillin down to 8 ppb, cephapirin down to 16 ppb, ceftiofur down to 100 ppb, cefquinone at less than 20 ppb, nafcillin down to 20 ppb and cefazolin down to 60 ppb in a milk sample.

This test is particularly suitable as a sorting test before milk lorries transfer their contents to the silos.

1.3.3. 9-minute test

Six samples of fresh milk are prepared, containing 0; 4; 6; 8; 10 and 12 ppb, respectively, of cehapirin. Each of these solutions is then analysed as follows.

An aliquot is taken of 200 μl of milk sample and 45.27 μl of solution prepared in Example 3.1.3, and is placed in a glass flask. This mixture is incubated at 47° C. for 7 minutes. An assay device is taken and is placed vertically in the glass flask such that the first end of the assay device is in contact with the mixture in such a way that the second end rests on the wall of the glass flask. The mixture is allowed to migrate over the assay device while the assembly is incubated at 47° C. for 2 minutes.

Table 3 below shows the results obtained for the 6 samples tested. An intensity value ranging from 0 to 10 is awarded to the bands detected, the value 10 being given to the most intense band and the value 0 being given to the least intense band. On this scale, a value of 6 is awarded to the reference band. The intensity of the signal observed in the first band is inversely proportional to the quantity of cephapirin present in the sample.

TABLE 3

| | |
|---|---|
| Cephapirin | Intensity |

| (ppb) | 1st band | 2nd band |
|---|---|---|
| 0 | 10 | 6 |
| 4 | 6 | 6 |
| 6 | 5 | 6 |
| 8 | 4 | 6 |
| 10 | 3 | 6 |
| 12 | 3 | 6 |

In this example, the test is considered to be positive when the first band has an intensity which is lower than that of the second band. The results shown in Table 3 indicate that this test makes it possible to detect in 9 minutes down to 6 ppb of cephapirin in a milk sample.

Assays were also carried out with other β-lactam ring antibiotics under the same conditions. This test, carried out in 9 minutes. makes it possible to detect penicillin G down to 3 ppb, amoxicillin down to 4 ppb, ampicillin down to 4 ppb, cloxacillin down to 4 ppb, dicloxacillin down to 8 ppb, oxacillin down to 8 ppb, ceftiofur down to 80 ppb, cefquinone at less than 20 ppb, nafcillin down to 20 ppb and cefazolin down to 45 ppb in a milk sample.

This test, carried out in 9 minutes, therefore allows detection of all the antibiotics which are at present monitored by the European authorities and does so down to the legal limits imposed by these authorities.

1.3.4 20-minute test

Six samples of fresh milk are prepared, containing 0; 20; 30; 40; 50 and 60 ppb, respectively, of ceftiofur. Each of these solutions is then analysed as follows.

An aliquot is taken of 200 μl of milk sample and 45.27 μl of solution prepared in Example 3.1.3, and is placed in a glass flask. This mixture is incubated at 47° C. for 18 minutes. An assay device is taken and is placed vertically in the glass flask such that the first end of the assay device is in contact with the mixture in such a way that the second end rests on the wall of the glass flask. The mixture is allowed to migrate over the assay device while the assembly is incubated at 47° C. for 2 minutes.

Table 4 below shows the results obtained for the 6 samples tested. An intensity value ranging from 0 to 10 is awarded to the bands detected, the value 10 being given to the most intense band and the value 0 being given to the least intense band. On this scale, a value 6 is awarded to the reference band. The intensity of the signal observed in the first detection band is inversely proportional to the quantity of ceftiofur present in the sample.

TABLE 4

| | |
|---|---|
| Ceftiofur | Intensity |

| (ppb) | 1st band | 2nd band |
|---|---|---|
| 0 | 10 | 6 |
| 20 | 6 | 6 |
| 30 | 5 | 6 |
| 40 | 4 | 6 |
| 50 | 3 | 6 |
| 60 | 3 | 6 |

In this example, the test is considered to be positive when the first band has an intensity which is lower than that of the second band. The results shown in Table 4 indicate that this test makes it possible to detect in 20 minutes down to 30 ppb of ceftiofur in a milk sample.

This 20-minute test therefore allows detection in a single test of all antibiotics currently monitored by the European and American authorities and does so down to the legal limits imposed by these authorities.

### EXAMPLE 4

### Use of an Assay Device in a Plastic Case

An assay device as described in Example 1.6. is used. In this instance, bringing the sample into contact with the assay device is carried out by depositing the incubated mixture in the basin-shaped opening provided for this purpose.

1. Assay device which allows the presence of analytes to be detected in a liquid dairy product by tangential capillary migration of the said dairy product, comprising a solid support (1) which has a first and a second end and on which the following membranes are fixed in succession starting from the first end:
a membrane (2) allowing the analysed liquid to be purified,
a membrane (3) on which one or more capture substances are immobilized, and
an absorbent membrane (4),
characterized in that the membrane (2) is capable of retaining the substances present in the dairy product which prevent the analytes, which may be present in the dairy product and the detection reagents used in accordance with the practised method, from migrating over the assay device during the tangential capillary migration of the sample after the first end of the assay device has been soaked in the analysed dairy product.

2. Device according to claim 1, characterized in that the membrane (2) is the Leukosorb membrane.

3. Assay device according to claim 1 or 2, characterized in that it additionally possesses a membrane (5) on which at least one detection reagent has been deposited, the membrane (5) being situated before the membrane (3).

4. Assay device according to claims 1 to 3, characterized in that the membranes are covered fully or partially by an adhesive plastic film (6).

5. Assay device according to claim 4, characterized in that the plastic film (6) does not cover the first few millimeters of the assay device.

6. Assay device according to claims 1 to 4, characterized in that it is located in a plastic box having an aperture in the form of a basin above the membrane (2) and a window aperture above the membrane (3).

7. Process for detecting analytes in a liquid dairy product, using an assay device according any one of claims 1 to 6, and detection reagents, and comprising the following steps:
a) the bringing into contact of a defined volume of dairy product with the assay device according to the present invention, this contact taking place at the first end of the assay device,
b) the tangential migration, by capillarity, of the dairy product over the assay device such that the analytes and the detection reagents which may be present in the dairy product pass gradually over the membrane (2), then the membrane (3), and such that the constituents of the dairy product which are not stopped by the membranes (2) and (3) end up in the membrane (4), and
c) the determination of a fixation on the membrane (3).

8. Process according to claim 7, characterized in that the detection reagents comprise at least one substance capable of recognizing specifically the analyte or an analogue substance of this analyte and at least one labelling agent.

9. Process according to claim 8, characterized in that at least one substance capable of recognizing specifically the analyte or an analogue substance of this analyte is coupled with at least one labelling agent.

10. Process according to claim 8 or 9, characterized in that the labelling agent is fluorescent, particulate, radioactive, luminescent or enzymatic.

11. Process according to claims 7 to 10, characterized in that at least one detection reagent is added before step a).

12. Process according to claim 11, characterized in that the mixture prepared before step a) is maintained under incubating conditions which allow one of the detection reagents to form a stable and essentially irreversible complex with the analyte or an analogue substance of this analyte.

13. Process according to claims 8 to 12, characterized in that the detection reagents additionally comprise at least one reference substance.

14. Process according to claims 7 to 13, characterized in that the analyte is a tetracyclin, such as tetracyclin, oxytetracyclin or chlortetracyclin.

15. Process according to claims 7 to 13, characterized in that the analyte is, on the one hand, an exogenous protein of the liquid dairy product, such as for example a foreign protein, or, on the other hand, an endogenous protein of the dairy product, for example an hormone such as progesterone or the growth hormone.

16. Process according to claims 7 to 13, characterized in that the analyte is an antibiotic being a β-lactam ring.

17. Process according to claims 7 to 13, characterized in that the analyte is selected from the group consisting of benzylpenicillin (penicillin G), ampicillin, amoxicillin, carbenicillin, methycillin, cloxacillin, 6-APA, monolactam, aztreonam, mecillinam, cephalexin, cephaloglycine, cephaloridine, nitrocephin, cefatoxime, defuroxime, ceftiofur, cephapirin, 7-ACA.

18. Process according to claims 8 to 17, characterized in that the substance capable of recognizing specifically the analyte or an analogue substance of the analyte is selected from receptors capable of forming a stable and essentially irreversible complex with the analyte or an analogue substance of the analyte and monoclonal or polyclonal antibodies specific for the analyte or for an analogue substance of the analyte.

19. Process according to claim 18, characterized in that the substance capable of recognizing specifically the analyte or an analogue substance of the analyte is a receptor obtained from an antibiotic-sensitive microorganism, such as the receptors obtained from Bacillus, Streptococcus or Actinomycetes species.

20. Process according to claims 18 or 19, characterized in that the substance capable of recognizing specifically the analyte or an analogue substance of the analyte is a receptor sensitive to antibiotics having a β-lactam ring, obtained from Bacillus licheniformis.

21. Process according to claim 20, characterized in that the receptor sensitive to antibiotics having a β-lactam ring and obtained from Bacillus licheniformis is the receptor BlaR or the receptor BlaR-CTD.

22. Process for preparing an assay device according to any one of claims 1 to 6, characterized in that cards are prepared from the solid support (1) covered with an adhesive and from membranes by means of a laminator, in that the capture solutions are deposited on the membrane (3) and in that, the cards are then cut into strips, each of these strips constituting an assay device.

23. Assay kit comprising an assay device according to claims 1 to 6.
